(19) United States
(12) Patent Application Publication
(10) Pub. No.: US 2023/0312767 A1
(43) Pub. Date: Oct. 5, 2023

(54) SYNERGISTIC ANTIFOULANT COMPOSITIONS AND METHODS OF USING THE SAME

(71) Applicant: ECOLAB USA INC., St. Paul, MN (US)

(72) Inventors: Zhenxing XI, Katy, TX (US); Jonathan MASERE, Richmond, TX (US); Ramon COLORADO, JR., Stafford, TX (US)

(73) Assignee: ECOLAB USA INC., St. Paul, MN (US)

(21) Appl. No.: 18/191,189

(22) Filed: Mar. 28, 2023

Related U.S. Application Data
(60) Provisional application No. 63/326,360, filed on Apr. 1, 2022.

(51) Int. Cl. C08F 2/00; C07D 295/24

(52) U.S. Cl. CPC C08F 2/005; C07D 295/24

(57) ABSTRACT

Polymerization inhibitor compositions are provided. The polymerization inhibitor compositions include at least a first inhibitor compound having a stable nitroxide radical and a second inhibitor compound having a hydroxylamine. Methods of inhibiting the polymerization of monomers using the compositions of the disclosure are also provided. The methods of inhibiting polymerization of monomers include a step of adding a composition of the disclosure to the monomer. In some instances, the monomer is an ethylenically unsaturated monomer. Such ethylenically unsaturated monomers include, but are not limited to, vinyl acetate, acrylonitrile, acrylates, methacrylates, 1,3-butadiene, styrene, isoprene, (meth)acrylic acid, and combinations thereof. Methods of preparing the polymerization inhibitors and compositions of the disclosure are also provided.

### FIELD OF THE INVENTION

The present disclosure generally relates to compositions that include a blend of polymerization inhibitors and methods of using the same. More particularly, the present disclosure relates to compositions that include at least one compound having a stable nitroxide radical and at least one compound having a hydroxylamine, useful for inhibiting polymerization of ethylenic unsaturated monomers. The present disclosure further relates to methods of preparing compounds having a stable nitroxide radical, as well as methods of preparing compounds having a hydroxylamine.

### BACKGROUND

The manufacture of ethylenically unsaturated monomers typically comprises three stages: reaction, recovery, and purification. Distillation operations at elevated temperatures are often involved in the recovery and the purification stages. Ethylenically unsaturated monomers, such as vinyl acetate, acrylate, and methacrylate monomers, can be present in processing streams or in refined products made by various chemical industrial processes. However, these monomer types may undesirably polymerize through radical polymerization especially at elevated temperature and when polymerization initiators are present. As a result, solid deposits of polymer can form on the surface of the process equipment during industrial manufacture, processing, handling, or storage. The resulting polymers can be problematic and lead to equipment “fouling” and product contamination. Accordingly, this can necessitate treating the apparatus to remove the polymer, or may necessitate processing steps to remove the polymer from compositions streams or stored compositions. These undesirable polymerization reactions result in a loss in production efficiency because they consume valuable reagents and additional steps may be required to clean equipment and/or to remove the undesired polymers.

The premature polymerization of these monomers is generally controlled by dosing polymerization inhibitors capable of reducing the premature polymerization of the monomers. Conventional polymerization inhibitors include stable free radicals that can effectively scavenge carbon-centered radicals. Conventional polymerization inhibitors, such as 4-hydroxy-2,2,6,6-tetramethylpiperidin-1-oxyl (HTEMPO) and 4-oxo-2,2,6,6-tetramethylpiperidin-1-oxyl (OTEMPO), generally degrade and lose their efficacy as polymerization inhibitors under acidic environments. Thus, there is a need to develop new polymerization inhibitors, particularly inhibitors that are stable under acidic conditions.

### BRIEF SUMMARY

Compositions for inhibiting the polymerization of monomers are disclosed herein. The compositions include a first inhibitor compound having a stable nitroxide radical and a second inhibitor compound having a hydroxylamine. In some embodiments, the compositions are useful for inhibiting the polymerization of ethylenically unsaturated monomers including vinyl acetate, acrylonitrile, acrylates, methacrylates, 1,3-butadiene, styrene, isoprene, (meth)acrylic acid, and combinations thereof.

In some embodiments, the compositions of the disclosure demonstrate synergy with respect to their ability to inhibit polymerization of a monomer. For example, in some embodiments, the compositions of the disclosure demonstrate greater polymerization inhibition than the individual components present within the composition, controlling for the dosage of the active components.

In some embodiments, the compositions of the disclosure are active even under acidic conditions, unlike conventional polymerization inhibitors known in the art. Thus, in some embodiments, the compositions of the disclosure also include one or more acids.

Methods of inhibiting the polymerization of a monomer are also disclosed herein. The methods of inhibiting the polymerization of a monomer include the step of adding a composition of the disclosure to the monomer. Addition of the polymerization inhibitor composition of the disclosure to the monomer inhibits polymerization of the monomer.

Processes for preparing the polymerization inhibitors and compositions of the disclosure are also disclosed herein. The processes for preparing a polymerization inhibitor include the step of treating a compound of formula (IIIa) with a compound of formula (IIIb) within a solution to afford the polymerization inhibitor compound.

The foregoing has outlined rather broadly the features and technical advantages of the present disclosure in order that the detailed description that follows may be better understood. Additional features and advantages of the disclosure will be described hereinafter that form the subject of the claims of this application. It should be appreciated by those skilled in the art that the conception and the specific embodiments disclosed may be readily utilized as a basis for modifying or designing other embodiments for carrying out the same purposes of the present disclosure. It should also be realized by those skilled in the art that such equivalent embodiments do not depart from the spirit and scope of the disclosure as set forth in the appended claims.

### BRIEF DESCRIPTION OF THE DRAWINGS

A detailed description of the invention is hereafter described with specific reference being made to the drawings.

FIG. 1 shows gelation test results of acrylic acid treated with Formulation 2 or Formulation 4 at a dosage of 3.2 ppm to 3.3 ppm. The results are compared to gelation test results of acrylic acid treated with 4-hydroxy-2,2,6,6-tetramethylpiperidin-1-oxyl (HTEMPO) or 4-oxo-2,2,6,6-tetramethylpiperidin-1-oxyl (OTEMPO).

FIG. 2 shows gelation test results of vinyl acetate in the presence of acetic acid treated with Formulation 1 or Formulation 4 at a dosage of about 50 ppm. The results are compared to gelation test results of acrylic acid treated with HTEMPO or OTEMPO.

### DETAILED DESCRIPTION

Various embodiments of the present disclosure are described below. The relationship and functioning of the various elements of the embodiments may better be understood by reference to the following detailed description. However, embodiments are not limited to those explicitly described herein and it should be understood that, in certain instances, details may have been omitted that are not necessary for an understanding of the embodiments disclosed herein, such as—for example—conventional synthesis and/or formulation.

The present disclosure relates to compositions that include a blend of polymerization inhibitors and methods of using the same to inhibit the polymerization of ethylenic unsaturated monomers. Polymerization inhibitor compositions of the present disclosure include at least one compound having a stable nitroxide radical and at least one compound having a hydroxylamine. The polymerization inhibitor compositions can be blends of multiple components, including components in addition to the aforementioned compounds having a stable nitroxide radical and a hydroxylamine.

The present disclosure further relates to methods of preparing compounds having a stable nitroxide radical, as well as methods of preparing compounds having a hydroxylamine. Any of the presently disclosed polymerization inhibitor compositions are effective in scavenging free radicals that would otherwise cause the initiation and propagation of a polymerization reaction involving ethylenic unsaturated monomers.

A “polymerization inhibitor,” in the presence of polymerizable monomers, inhibits the formation of a polymer from those monomers during the induction time. After the induction time has lapsed, the polymer's formation occurs at substantially the same rate that it would form at in the absence of the polymerization inhibitor.

Polymerization inhibitors and polymerization retarders can be considered generally as “antipolymerants” which are compounds that can inhibit or reduce the formation of polymers from one or more radically polymerizable compounds.

The term “fouling” refers to the formation of polymers, prepolymers, oligomer and/or other materials, which would become insoluble in and/or precipitate from a stream and deposit on equipment under the conditions of operation of the equipment. In turn, the inhibitor, retarder, and amine stabilizer components and compositions of the disclosure can be referred to as “antifouling” as they inhibit or reduce such formation.

### Compositions of the Disclosure

The present disclosure relates to compositions for inhibiting monomer polymerization where the compositions include a first inhibitor compound having a stable nitroxide radical and a second inhibitor compound having a hydroxylamine. In some embodiments, the compositions are for inhibiting monomer polymerization, where the monomer is an ethylenic unsaturated monomer. For example, the compositions of the disclosure are useful for inhibiting polymerization of ethylenic unsaturated monomers including, but not limited to, vinyl acetate, acrylonitrile, acrylates, methacrylates, 1,3-butadiene, styrene, isoprene, (meth)acrylic acid, and combinations thereof.

In some embodiments, the first inhibitor compound having a stable nitroxide radical is a compound of formula (I):

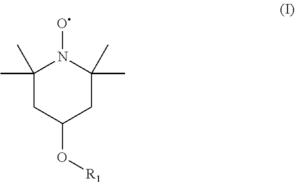

where R₁ is selected from H, C₁-C₂₂ alkyl, C₁-C₂₂ alkenyl, C₁-C₂₂ alkynyl, C₁-C₂₂ cycloalkyl, aryl, —C₁-C₂₂ alkylene aryl, —C(O)(C₁-C₂₂ alkyl), —C(O)(C₁-C₂₂ alkenyl), —C(O)(C₁-C₂₂ alkynyl), —C(O)(C₁-C₂₂ cycloalkyl), —C(O)(aryl), and —C(O)(C₁-C₂₂ alkylene aryl), where the cycloalkyl and aryl are optionally substituted with one or more C₁-C₂₂ alkyl, C₁-C₂₂ alkenyl, C₁-C₂₂ alkynyl, or aryl.

In certain embodiments, R₁ is selected from —C(O)(C₁-C₂₂ alkyl), —C(O)(C₁-C₂₂ alkenyl), —C(O)(C₁-C₂₂ alkynyl), —C(O)(C₁-C₂₂ cycloalkyl), —C(O)(aryl), and —C(O)(C₁-C₂₂ alkylene aryl), wherein the cycloalkyl and aryl are optionally substituted with one or more C₁-C₂₂ alkyl, C₁-C₂₂ alkenyl, C₁-C₂₂ alkynyl, and aryl.

In some embodiments, R₁ is H. In some embodiments, R₁ is C₁-C₂₂ alkyl. In some embodiments, R₁ is C₁-C₂₂ alkenyl. In some embodiments, R₁ is C₁-C₂₂ alkynyl. In some embodiments, R₁ is C₁-C₂₂ cycloalkyl, where the cycloalkyl is optionally substituted with one or more C₁-C₂₂ alkyl, C₁-C₂₂ alkenyl, C₁-C₂₂ alkynyl, or aryl. In some embodiments, R₁ is aryl, where the aryl is optionally substituted with one or more C₁-C₂₂ alkyl, C₁-C₂₂ alkenyl, C₁-C₂₂ alkynyl, or aryl. In some embodiments, R₁ is —C₁-C₂₂ alkylene aryl, where the aryl is optionally substituted with one or more C₁-C₂₂ alkyl, C₁-C₂₂ alkenyl, C₁-C₂₂ alkynyl, or aryl. In some embodiments, R₁ is —C(O)(C₁-C₂₂ alkyl). In some embodiments, R₁ is —C(O)(C₁-C₂₂ alkyl). In some embodiments, R₁ is —C(O)(C₁-C₆ alkyl). In some embodiments, R₁ is —C(O)(methyl). In some embodiments, R₁ is —C(O)(ethyl). In some embodiments, R₁ is —C(O)(propyl). In some embodiments, R₁ is —C(O)(butyl). In some embodiments, R₁ is —C(O)(C₁-C₂₂ alkenyl). In some embodiments, R₁ is —C(O)(C₁-C₂₂ alkynyl). In some embodiments, R₁ is —C(O)(C₁-C₂₂ cycloalkyl), where the cycloalkyl is optionally substituted with one or more C₁-C₂₂ alkyl, C₁-C₂₂ alkenyl, C₁-C₂₂ alkynyl, or aryl. In some embodiments, R₁ is —C(O)(aryl), where the aryl is optionally substituted with one or more C₁-C₂₂ alkyl, C₁-C₂₂ alkenyl, C₁-C₂₂ alkynyl, or aryl. In some embodiments, R₁ is —C(O)(C₁-C₂₂ alkylene aryl), where the aryl is optionally substituted with one or more C₁-C₂₂ alkyl, C₁-C₂₂ alkenyl, C₁-C₂₂ alkynyl, or aryl.

In some embodiments, the second inhibitor compound having a hydroxylamine is a compound of formula (II):

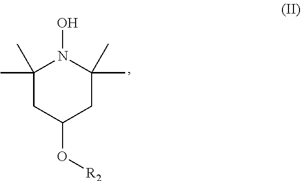

where R₂ is selected from H, C₁-C₂₂ alkyl, C₁-C₂₂ alkenyl, C₁-C₂₂ alkynyl, C₁-C₂₂ cycloalkyl, aryl, —C₁-C₂₂ alkylene aryl, —C(O)(C₁-C₂₂ alkyl), —C(O)(C₁-C₂₂ alkenyl), —C(O)(C₁-C₂₂ alkynyl), —C(O)(C₁-C₂₂ cycloalkyl), —C(O)(aryl), and —C(O)(C₁-C₂₂ alkylene aryl), wherein the cycloalkyl and aryl are optionally substituted with one or more C₁-C₂₂ alkyl, C₁-C₂₂ alkenyl, C₁-C₂₂ alkynyl, or aryl.

In certain embodiments, R₂ is —C(O)(C₁-C₂₂ alkyl), —C(O)(C₁-C₂₂ alkenyl), —C(O)(C₁-C₂₂ alkynyl), —C(O)(C₁-C₂₂ cycloalkyl), —C(O)(aryl), and —C(O)(C₁-C₂₂ alkylene aryl), where the cycloalkyl and aryl are optionally substituted with one or more C₁-C₂₂ alkyl, C₁-C₂₂ alkenyl, C₁-C₂₂ alkynyl, or aryl.

In some embodiments, R₂ is H. In some embodiments, R₂ is C₁-C₂₂ alkyl. In some embodiments, R₂ is C₁-C₂₂ alkenyl. In some embodiments, R₂ is C₁-C₂₂ alkynyl. In some embodiments, R₂ is C₁-C₂₂ cycloalkyl, where the cycloalkyl is optionally substituted with one or more C₁-C₂₂ alkyl, C₁-C₂₂ alkenyl, C₁-C₂₂ alkynyl, or aryl. In some embodiments, R₂ is aryl, where the aryl is optionally substituted with one or more C₁-C₂₂ alkyl, C₁-C₂₂ alkenyl, C₁-C₂₂ alkynyl, or aryl. In some embodiments, R₂ is —C₁-C₂₂ alkylene aryl, where the aryl is optionally substituted with one or more C₁-C₂₂ alkyl, C₁-C₂₂ alkenyl, C₁-C₂₂ alkynyl, or aryl. In some embodiments, R₂ is —C(O)(C₁-C₂₂ alkyl). In some embodiments, R₂ is —C(O)(C₁-C₁₂ alkyl). In some embodiments, R₂ is —C(O)(C₁-C₂₂ alkyl). In some embodiments, R₂ is —C(O)(methyl). In some embodiments, R₂ is —C(O)(ethyl). In some embodiments, R₂ is —C(O)(propyl). In some embodiments, R₂ is —C(O)(butyl). In some embodiments, R₂ is —C(O)(C₁-C₂₂ alkenyl). In some embodiments, R₂ is —C(O)(C₁-C₂₂ alkynyl). In some embodiments, R₂ is —C(O)(C₁-C₂₂ cycloalkyl), where the cycloalkyl is optionally substituted with one or more C₁-C₂₂ alkyl, C₁-C₂₂ alkenyl, C₁-C₂₂ alkynyl, or aryl. In some embodiments, R₂ is —C(O)(aryl), where the aryl is optionally substituted with one or more C₁-C₂₂ alkyl, C₁-C₂₂ alkenyl, C₁-C₂₂ alkynyl, or aryl. In some embodiments, R₂ is —C(O)(C₁-C₂₂ alkylene aryl), where the aryl is optionally substituted with one or more C₁-C₂₂ alkyl, C₁-C₂₂ alkenyl, C₁-C₂₂ alkynyl, or aryl.

In certain embodiments, the compositions of the disclosure include first and second inhibitor compounds of formula (I) and (II), respectively, where R₁ and R₂ are the same. For example, in some embodiments, the compositions of the disclosure include first and second inhibitor compounds of formula (I) and (II), respectively, where R₁ and R₂ are each, independently, —C(O)(C₁-C₂₂ alkyl). In certain embodiments, the compositions of the disclosure include first and second inhibitor compounds of formula (I) and (II), respectively, where R₁ and R₂ are different.

In some embodiments, the first inhibitor compound having a stable nitroxide radical is a compound selected from the group consisting of:

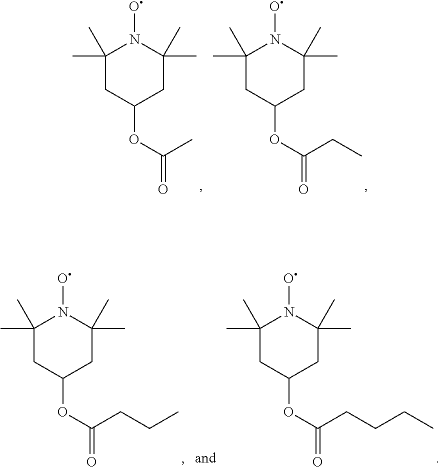

In certain embodiments, the first inhibitor compound having a stable nitroxide radical is a compound selected from:

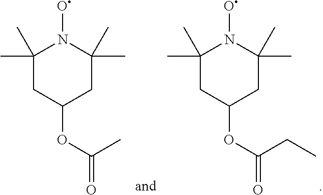

In some embodiments, the first inhibitor compound having a stable nitroxide radical is:

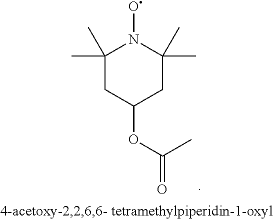

In some embodiments, the first inhibitor compound having a stable nitroxide radical is:

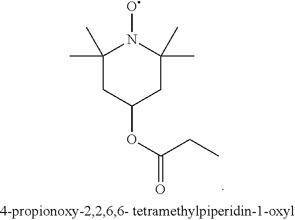

In some embodiments, the first inhibitor compound having a stable nitroxide radical is:

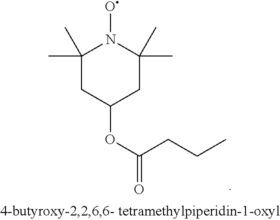

In some embodiments, the first inhibitor compound having a stable nitroxide radical is:

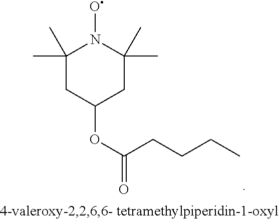

In some embodiments, the second inhibitor compound having a hydroxylamine is selected from the group consisting of:

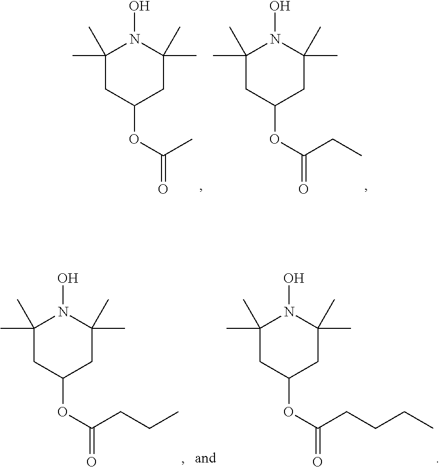

In some embodiments, the second inhibitor compound having a hydroxylamine is:

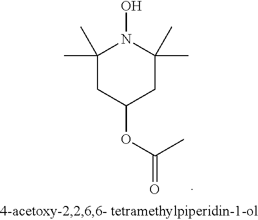

In some embodiments, the second inhibitor compound having a hydroxylamine is:

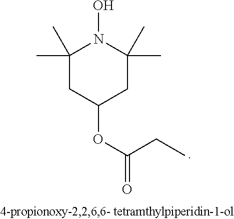 [SMILES: CCC(=O)OC1CC(C)(C)N(O)C(C)(C)C1]

In some embodiments, the second inhibitor compound having a hydroxylamine is:

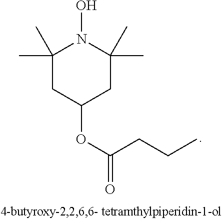 [SMILES: CCCC(=O)OC1CC(C)(C)N(O)C(C)(C)C1]

In some embodiments, the second inhibitor compound having a hydroxylamine is:

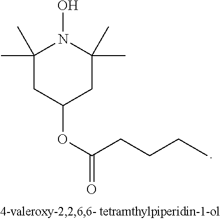 [SMILES: CCCCC(=O)OC1CC(C)(C)N(O)C(C)(C)C1]

The presently disclosed second inhibitor compound having a hydroxylamine has benefits over the corresponding nitroxide, such as the capability to provide additional polymerization inhibition, as will be more fully explained below. A general synthetic route to produce a hydroxylamine of a nitroxide is to reduce its corresponding nitroxide with a reducing reagent as follows:

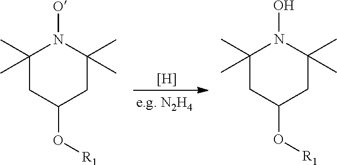

A hydroxylamine of a nitroxide has the potential to provide additional polymerization inhibition as compared to the corresponding nitroxide when carbon-centered and oxygen-centered radical initiators are present. This is explained as follows:

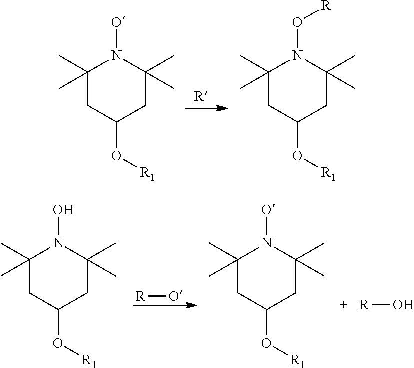

The hydroxylamine of a nitroxide is an excellent hydrogen donor due to its weak NO—H bond in the compound, and thus it is an efficient antioxidant. As an antioxidant, the hydroxylamine of a nitroxide easily reacts with oxygen-centered radicals, such as peroxide radicals, while it's converted to its corresponding nitroxide. Nitroxides are generally known as the most effective inhibitors because of their superior inhibiting capabilities through scavenging carbon-centered free radicals at a nearly diffusion controlled rate. This rate is several orders of magnitude faster than phenolic compounds. However, their kinetic superiority is not always advantageous. For instance, it may lose its superiority when oxygen-centered radicals are present as the predominant free radicals. Another issue of concern with a nitroxide is its consumption through non-inhibition and unwanted reactions with process stream components or other inhibitor additives. As a result, high nitroxide inhibitor dosages are often required for a given inhibition efficacy, thereby making their use economically unattractive or even infeasible.

In essence, each hydroxylamine of a nitroxide is equivalent to one hydrogen donor plus one nitroxide antipolymerant when oxygen-centered radicals and carbon-centered radicals are both present, which is an attractive incentive offered by the hydroxylamines of nitroxides. That is, one hydroxylamine of a nitroxide is able to eliminate one oxygen-centered radical and one carbon-centered radical whereas a nitroxide is only capable to eliminate a carbon-centered radical.

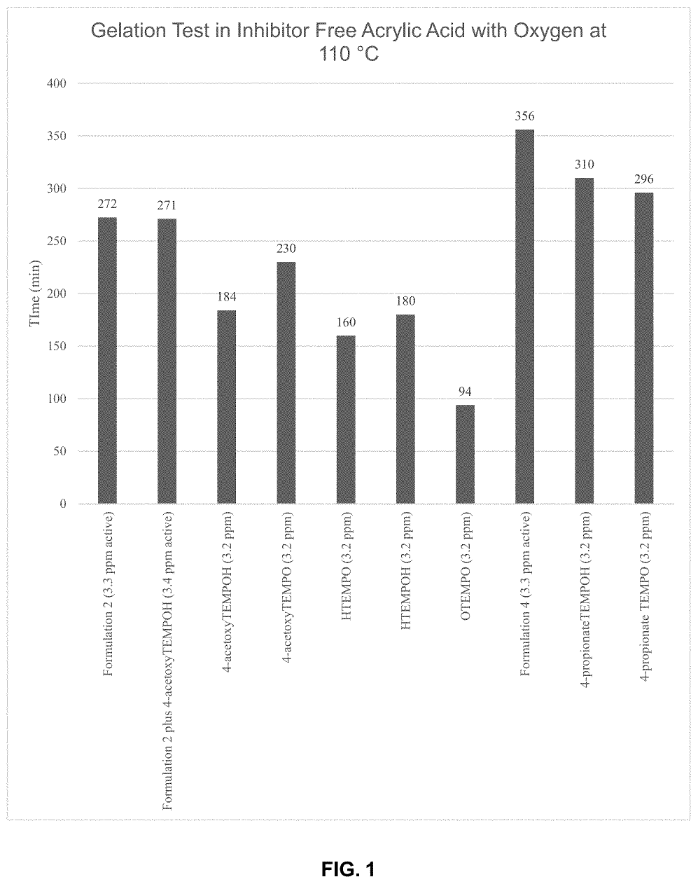

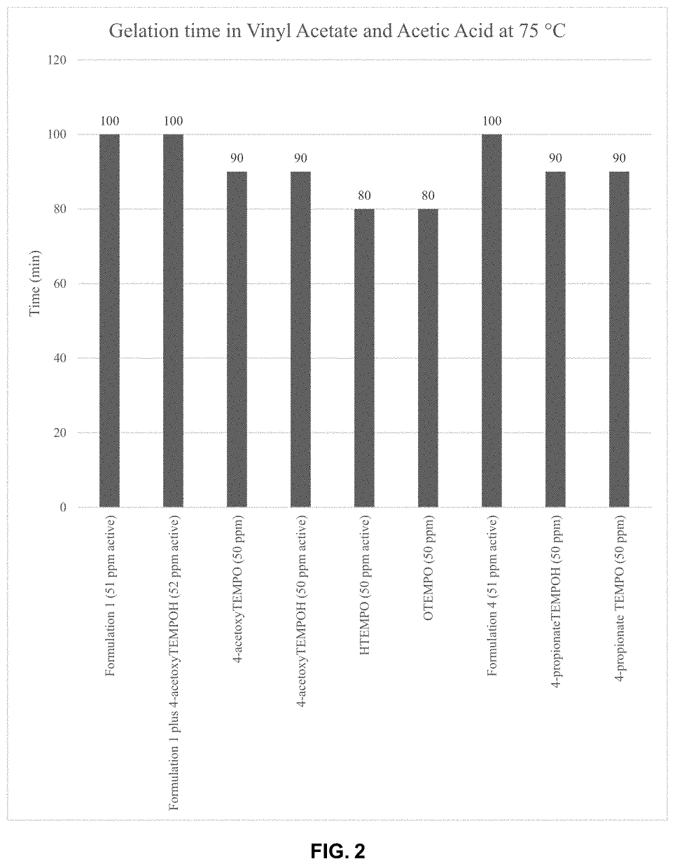

In some embodiments, the composition unexpectedly demonstrates synergy, where the combination of the first and second inhibitor compounds produces a greater degree of polymerization inhibition than would be expected for the combination. This unexpected synergy is demonstrated in the Examples presented herein, as well as in FIGS. 1 and 2.

In some embodiments, the first inhibitor compound having a stable nitroxide radical is present in the composition at a concentration of about 0.01% by weight to about 80% by weight. In some embodiments, the first inhibitor compound having a stable nitroxide radical is present in the composition at a concentration of about 0.01% by weight to about 70% by weight. In some embodiments, the first inhibitor compound having a stable nitroxide radical is present in the composition at a concentration of about 0.01% by weight to about 60% by weight. In some embodiments, the first inhibitor compound having a stable nitroxide radical is present in the composition at a concentration of about 0.01% by weight to about 50% by weight. In some embodiments, the first inhibitor compound having a stable nitroxide radical is present in the composition at a concentration of about 0.01% by weight to about 40% by weight. In some embodiments, the first inhibitor compound having a stable nitroxide radical is present in the composition at a concentration of about 0.01% by weight to about 30% by weight. In some embodiments, the first inhibitor compound having a stable nitroxide radical is present in the composition at a concentration of about 0.01% by weight to about 20% by weight. In some embodiments, the first inhibitor compound having a stable nitroxide radical is present in the composition at a concentration of about 0.01% by weight to about 10% by weight.

For example, in certain embodiments, the first inhibitor compound having a stable nitroxide radical is present in the composition at a concentration of about 0.01% by weight, about 0.1% by weight, about 1% by weight, about 5% by weight, about 10% by weight, about 15% by weight, about 20% by weight, about 25% by weight, about 30% by weight, about 35% by weight, about 40% by weight, about 45% by weight, about 50% by weight, about 55% by weight, about 60% by weight, about 65% by weight, about 70% by weight, about 75% by weight, or about 80% by weight.

In some embodiments, the second inhibitor compound having a hydroxylamine is present in the composition at a concentration of about 0.01% by weight to about 50% by weight. In some embodiments, the second inhibitor compound having a hydroxylamine is present in the composition at a concentration of about 0.01% by weight to about 40% by weight. In some embodiments, the second inhibitor compound having a hydroxylamine is present in the composition at a concentration of about 0.01% by weight to about 30% by weight. In some embodiments, the second inhibitor compound having a hydroxylamine is present in the composition at a concentration of about 0.01% by weight to about 20% by weight. In some embodiments, the second inhibitor compound having a hydroxylamine is present in the composition at a concentration of about 0.01% by weight to about 10% by weight.

For example, in certain embodiments, the second inhibitor compound having a hydroxylamine is present in the composition at a concentration of about 0.01% by weight, about 0.1% by weight, about 1% by weight, about 5% by weight, about 10% by weight, about 15% by weight, about 20% by weight, about 25% by weight, about 30% by weight, about 35% by weight, about 40% by weight, about 45% by weight, or about 50% by weight.

In some embodiments, a mole ratio of the first inhibitor compound having a stable nitroxide radical to the second inhibitor compound having a hydroxylamine is about 100:1 to about 1:100. In some embodiments, a mole ratio of the first inhibitor compound having a stable nitroxide radical to the second inhibitor compound having a hydroxylamine is about 90:1 to about 1:90. In some embodiments, a mole ratio of the first inhibitor compound having a stable nitroxide radical to the second inhibitor compound having a hydroxylamine is about 80:1 to about 1:80. In some embodiments, a mole ratio of the first inhibitor compound having a stable nitroxide radical to the second inhibitor compound having a hydroxylamine is about 70:1 to about 1:70. In some embodiments, a mole ratio of the first inhibitor compound having a stable nitroxide radical to the second inhibitor compound having a hydroxylamine is about 60:1 to about 1:60. In some embodiments, a mole ratio of the first inhibitor compound having a stable nitroxide radical to the second inhibitor compound having a hydroxylamine is about 50:1 to about 1:50. In some embodiments, a mole ratio of the first inhibitor compound having a stable nitroxide radical to the second inhibitor compound having a hydroxylamine is about 40:1 to about 1:40. In some embodiments, a mole ratio of the first inhibitor compound having a stable nitroxide radical to the second inhibitor compound having a hydroxylamine is about 30:1 to about 1:30. In some embodiments, a mole ratio of the first inhibitor compound having a stable nitroxide radical to the second inhibitor compound having a hydroxylamine is about 20:1 to about 1:20. In some embodiments, a mole ratio of the first inhibitor compound having a stable nitroxide radical to the second inhibitor compound having a hydroxylamine is about 10:1 to about 1:10. In some embodiments, a mole ratio of the first inhibitor compound having a stable nitroxide radical to the second inhibitor compound having a hydroxylamine is about 1:1.

In some embodiments, the composition also includes one or more additional compounds selected from the group consisting of 2,2,6,6-tetramethylpiperidin-1-oxyl; 2,2,6,6-tetramethylpiperidin-1-ol; 4-hydroxyl-2,2,6,6-tetramethylpiperidin-1-oxyl; 4-hydroxy-2,2,6,6-tetramethylpiperidin-1-ol; 4-oxo-2,2,6,6-tetramethylpiperidin-1-oxyl; 4-oxo-2,2,6,6-tetramethylpiperidin-1-ol; 4-acetoxy-2,2,6,6-tetramethylpiperidin-1-oxyl; 4-acetoxy-2,2,6,6-tetramethylpiperidin-1-ol; 4-propionoxy-2,2,6,6-tetramethylpiperidin-1-oxyl; 4-propionoxy-2,2,6,6-tetramethylpiperidin-1-ol; and bis((2,2,6,6-tetramethylpiperdin-1-oxyl)-4-yl) oxalate. In some embodiments, the composition also includes 2,2,6,6-tetramethylpiperidin-1-oxyl. In some embodiments, the composition also includes 2,2,6,6-tetramethylpiperidin-1-ol. In some embodiments, the composition also includes 4-hydroxyl-2,2,6,6-tetramethylpiperidin-1-oxyl. In some embodiments, the composition also includes 4-hydroxy-2,2,6,6-tetramethylpiperidin-1-ol. In some embodiments, the composition also includes 4-oxo-2,2,6,6-tetramethylpiperidin-1-oxyl. In some embodiments, the composition also includes 4-oxo-2,2,6,6-tetramethylpiperidin-1-ol. In some embodiments, the composition also includes 4-acetoxy-2,2,6,6-tetramethylpiperidin-1-oxyl. In some embodiments, the composition also includes 4-acetoxy-2,2,6,6-tetramethylpiperdin-1-ol. In some embodiments, the composition also includes 4-propionoxy-2,2,6,6-tetramethylpiperidin-1-oxyl. In some embodiments, the composition also includes 4-propionoxy-2,2,6,6-tetramethylpiperdin-1-ol. In some embodiments, the composition also includes bis((2,2,6,6-tetramethylpiperidin-1-oxyl)-4-yl) oxalate.

The composition may optionally also include one or more organic solvents. One of ordinary skill in the art will appreciate that there are many organic solvents that are compatible with the compositions of the disclosure. For example, in some embodiments, the one or more organic solvents are selected from vinyl acetate, dimethyl phthalate, dimethylformamide, toluene, xylene, highly aromatic naphtha, acetonitrile, ethyl acetate, acetone, dichloromethane, tetrahydrofuran, hexanes, dimethyl sulfoxide, N-methyl-2-pyrrolidone, and combinations thereof. In certain embodiments, the composition also includes vinyl acetate. In certain embodiments, the composition also includes dimethyl phthalate. In certain embodiments, the composition also includes dimethylformamide. In certain embodiments, the composition also includes toluene. In certain embodiments, the composition also includes xylene. In certain embodiments, the composition also includes highly aromatic naphtha. In certain embodiments, the composition also includes acetonitrile.

In some embodiments, the composition also includes one or more ethylenic unsaturated monomers. One of ordinary skill in the art will appreciate that there are many ethylenic unsaturated monomers that are compatible with the compositions of the disclosure. For example, in some embodiments, the one or more ethylenic unsaturated monomers are selected from vinyl acetate, acrylonitrile, acrylates, methacrylates, 1,3-butadiene, styrene, isoprene, (meth)acrylic acid, and combinations thereof. In certain embodiments, the composition also includes vinyl acetate. In certain embodiments, the composition also includes acrylonitrile. In certain embodiments, the composition also includes acrylates. In certain embodiments, the composition also includes methacrylates. In certain embodiments, the composition also includes 1,3-butadiene. In certain embodiments, the composition also includes styrene. In certain embodiments, the composition also includes isoprene. In certain embodiments, the composition also includes (meth)acrylic acid.

The compositions of the disclosure are stable and remain useful polymerization inhibitors even under acidic conditions. Thus, the compositions of the disclosure are useful for inhibiting the premature polymerization of monomers during manufacturing process, particularly those that are performed under acidic conditions. For example, the compositions of the disclosure are useful for preventing polymerization of acrylates, which may include, but are not limited to, acrylonitrile, acrylic acid, methyl methacrylic acid and its esters, and vinyl acetate.

In certain embodiments, the compositions of the disclosure are generally stable under acidic conditions, a significant improvement over conventional polymerization inhibitors known in the art. Thus, in some embodiments, the composition also includes one or more acids. For example, in some embodiments, the composition also includes one or more acids selected from the group consisting of mineral acids and carboxylic acids. Mineral acids include, but are not limited to, hydrochloric acid, hydrofluoric acid, hydrobromic acid, hydroiodic acid, nitric acid, phosphoric acid, sulfuric acid, boric acid, perchloric acid, and the like. Carboxylic acids include, but are not limited to, formic acid, acetic acid, propionic acid, butyric acid, valeric acid, caproic acid, ethanic acid, caprylic acid, undecylic acid, lauric acid, oxalic acid, malonic acid, succinic acid, glutaric acid, adipic acid, pimelic acid, suberic acid, and the like. In some embodiments, the compositions of the disclosure also include hydrochloric acid. In some embodiments, the compositions of the disclosure also include nitric acid. In some embodiments, the compositions of the disclosure also include phosphoric acid. In some embodiments, the compositions of the disclosure also include sulfuric acid. In some embodiments, the compositions of the disclosure also include acetic acid. In some embodiments, the compositions of the disclosure also include propionic acid. In some embodiments, the compositions of the disclosure also include butyric acid. In some embodiments, the compositions of the disclosure also include valeric acid.

In some embodiments, the composition also includes acetaldehyde.

The compositions of the disclosure have balanced partition coefficients between polar organic phases and polar phases. Thus, the compositions of the disclosure are also useful in butadiene extraction processes.

### Methods of Using the Compositions of the Disclosure

The present disclosure also relates to methods of inhibiting polymerization of monomers that include adding a composition of the disclosure to the monomer. In some aspects, an effective amount of the composition of the disclosure is added to the monomer, where an effective amount is any amount sufficient to inhibit the polymerization of the monomer.

In some aspects, the monomer is an ethylenic unsaturated monomer. In some aspects the monomer is an ethylenic unsaturated monomer selected from vinyl acetate, acrylonitrile, acrylates, methacrylates, 1,3-butadiene, styrene, isoprene, (meth)acrylic acid, and combinations thereof are disclosed. In some aspects, the methods disclosed herein are useful in inhibiting the polymerization of vinyl acetate. In some aspects, the methods disclosed herein are useful in inhibiting the polymerization of acrylonitrile. In some aspects, the methods disclosed herein are useful in inhibiting the polymerization of acrylates. In some aspects, the methods disclosed herein are useful in inhibiting the polymerization of methacrylates. In some aspects, the methods disclosed herein are useful in inhibiting the polymerization of 1,3-butadiene. In some aspects, the methods disclosed herein are useful in inhibiting the polymerization of styrene. In some aspects, the methods disclosed herein are useful in inhibiting the polymerization of isoprene. In some aspects, the methods disclosed herein are useful in inhibiting the polymerization of (meth)acrylic acid.

The composition of the disclosure can be added manually or automatically to the fluid. The composition can also be added continuously and/or intermittently. Automatic addition may be accomplished through the use of chemical injection pumps. The chemical injection pumps may be programmed to add particular amounts of the polymerization inhibitor composition, or any components thereof, at certain time intervals to the fluid. In alternate aspects, the chemical injection pumps can be manually controlled to add particular amounts of the polymerization inhibitor composition, or any components thereof, to the fluid. Addition of the presently disclosed polymerization inhibitor compositions to the monomer will thereby inhibit polymerization of the monomer.

In some aspects, the monomer is provided as a neat liquid. In other aspects, the monomer is provided within a solution, hereafter referred to as “the monomer solution”.

In some aspects, the monomer solution also includes one or more additional components selected from an acid, an organic solvent, water, and combinations thereof. For example, in some aspects, the monomer solution includes one or more organic solvents selected from vinyl acetate, dimethyl phthalate, dimethylformamide, toluene, xylene, highly aromatic naphtha, acetonitrile, ethyl acetate, acetone, dichloromethane, tetrahydrofuran, hexanes, dimethyl sulfoxide, N-methyl-2-pyrrolidone, and combinations thereof. In some aspects, the monomer solution includes one or more acids selected from hydrochloric acid, hydrofluoric acid, hydrobromic acid, hydroiodic acid, nitric acid, phosphoric acid, sulfuric acid, boric acid, perchloric acid, formic acid, acetic acid, propionic acid, butyric acid, valeric acid, caproic acid, ethanic acid, caprylic acid, undecylic acid, lauric acid, oxalic acid, malonic acid, succinic acid, glutaric acid, adipic acid, pimelic acid, and suberic acid. In some aspects, the monomer solution includes water.

In some aspects, the monomer solution has a pH value of about 1 to about 7. In some aspects, the monomer solution has a pH value of about 1 to about 6. In some aspects, the monomer solution has a pH value of about 2 to about 6. In some aspects, the monomer solution has a pH value of about 3 to about 6. In some aspects, the monomer solution has a pH value of about 4 to about 6. In some aspects, the monomer solution has a pH value of about 5 to about 6.

In some aspects, the composition is added to the monomer such that a concentration of the first inhibitor compound is about 0.1 ppm to about 10,000 ppm. In some aspects, the composition is added to the monomer such that a concentration of the first inhibitor compound is about 0.1 ppm to about 5,000 ppm. In some aspects, the composition is added to the monomer such that a concentration of the first inhibitor compound is about 0.1 ppm to about 1,000 ppm. In some aspects, the composition is added to the monomer such that a concentration of the first inhibitor compound is about 0.1 ppm to about 500 ppm.

In some aspects, the composition is added to the monomer such that a concentration of the second inhibitor compound is about 0.1 ppm to about 10,000 ppm. In some aspects, the composition is added to the monomer such that a concentration of the second inhibitor compound is about 0.1 ppm to about 5,000 ppm. In some aspects, the composition is added to the monomer such that a concentration of the second inhibitor compound is about 0.1 ppm to about 1,000 ppm. In some aspects, the composition is added to the monomer such that a concentration of the second inhibitor compound is about 0.1 ppm to about 500 ppm.

The methods of the disclosure are useful for inhibiting the premature polymerization of monomers during manufacturing process, particularly those that are performed under acidic conditions. For example, the methods of the disclosure are useful for preventing polymerization of acrylates, which may include, but are not limited to, acrylonitrile, acrylic acid, methyl methacrylic acid and its esters, and vinyl acetate.

The methods of the disclosure are also useful for preventing the premature polymerization of styrene during manufacturing and purification processes.

The methods of the disclosure are also useful in butadiene extraction processes. This utility stems from the balanced partition coefficients between polar organic phases and organic phases.

### Processes for Preparing Polymerization Inhibitors of the Disclosure

The present disclosure also relates to processes for preparing a compound of formula (III):

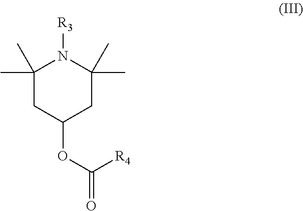

wherein:
R₃ is —O⋅ or —OH; and
R₄ is C₁-C₂₂ alkyl, C₁-C₂₂ alkenyl, C₁-C₂₂ alkynyl, C₁-C₂₂ cycloalkyl, aryl, and C₁-C₂₂ alkylene aryl, wherein the cycloalkyl and aryl are optionally substituted with one or more C₁-C₂₂ alkyl, C₁-C₂₂ alkenyl, C₁-C₂₂ alkynyl, or aryl.

In some aspects, the process for preparing a compound of formula (III) includes treating a compound of formula (IIIa):

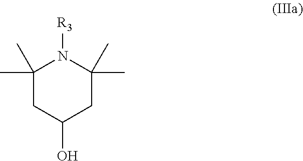

with a compound of (IIIb):

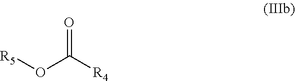

wherein R₅ is C₁-C₂₂ alkyl or C₁-C₂₂ alkenyl,
   within a solution, to afford the compound of formula (III).

In some aspects, R₃ is —O⋅. In some aspects, R₃ is —OH.

In some aspects, R₄ is C₁-C₂₂ alkyl. In some aspects, R₄ is C₁-C₁₂ alkyl. In some aspects, R₄ is C₁-C₆ alkyl. In some aspects, R₄ is ethyl. In some aspects, R₄ is methyl.

In one aspect, R₃ is —O⋅ and R₄ is methyl. In another aspect, R₃ is —O⋅ and R₄ is ethyl. In another aspect, R₃ is —OH and R₄ is methyl. In another aspect, R₃ is —OH and R₄ is ethyl.

In some aspects, R₅ is C₁-C₂₂ alkyl. In some aspects, R₅ is C₁-C₁₂ alkyl. In some aspects, R₅ is C₁-C₆ alkyl.

In some aspects R₅ is C₁-C₂₂ alkenyl. In some aspects, R₅ is C₁-C₁₂ alkenyl. In some aspects, R₅ is C₁-C₆ alkenyl. In some aspects, R₅ is C₂ alkenyl.

In some aspects, the process for preparing a compound of formula (III) includes treating a compound of formula (IIIa):

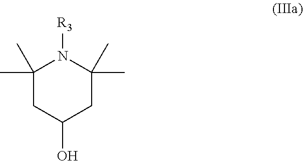

with a compound of (IIIc):

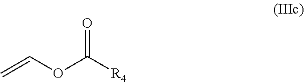

within a solution, to afford the compound of formula (III).

In some aspects, the compound of formula (IIIa) is treated with the compound of formula (IIIb) in the presence of a catalyst and heat. In some aspects, the compound of formula (IIIa) is treated with the compound of formula (IIIc) in the presence of a catalyst and heat. One of ordinary skill will appreciate that there are many appropriate catalysts that can be used to form a compound of formula (III) by treating a compound of formula (IIIa) with a compound of formula (IIIb) or (IIIc). For example, in some aspects, the catalyst is an amine-containing compound. In certain aspects, the catalyst is 4-dimethylaminopyridine, also known as DMAP. In some aspects, the solution of the compound of formula (IIIa) and (IIIb), or (IIIc), is heated to a temperature of about 50° C. to about 100° C. In some aspects, the solution is heated to a temperature of about 50° C. to about 85° C.

In some aspects, the process for preparing a compound of formula (III) also includes purging the solution with a stream of nitrogen. In some aspects, the step of purging the solution with a stream of nitrogen is performed concurrently with the step of treating a compound of formula (IIIa) with a compound of formula (IIIb) to afford the compound of formula (III). In some aspects, the step of purging the solution with a stream of nitrogen is performed concurrently with the step of treating a compound of formula (IIIa) with a compound of formula (IIIc) to afford the compound of formula (III). In some aspects, the step of purging the solution with a stream of nitrogen is performed after the step of treating a compound of formula (IIIa) with a compound of formula (IIIb) to afford the compound of formula (III). In some aspects, the step of purging the solution with a stream of nitrogen is performed after the step of treating a compound of formula (IIIa) with a compound of formula (IIIc) to afford the compound of formula (III). Without being bound by theory, the step of purging the solution with a stream of nitrogen may be useful in removing certain reaction byproducts that pushes the reaction equilibrium towards formation of the compound of formula (III).

### EXAMPLES

### Example 1—Preparation of Formulation 1

A composition of the disclosure, hereafter referred to as Formulation 1, was prepared using the following procedure.

A reaction vessel was charged with 206 g 4-hydroxy-2,2,6,6-tetramethylpiperidin-1-oxyl (HTEMPO) and 290 g vinyl acetate. The resulting solution was subsequently agitated until all of the HTEMPO dissolved, and the resulting mixture was heated to 65° C. This was followed by the addition of 1.5 g 4-dimethylaminopyridine (DMAP). The reaction temperature was maintained at 65° C. and the mixture was stirred for 30 minutes. The reaction temperature was subsequently increased to 75° C. and the solution was stirred for an additional 30 minutes. 2.4 g of DMAP were then added slowly to the reaction mixture, which was then stirred for an additional 1 hour, maintaining the reaction temperature at 75° C. The reaction mixture was then heated to a temperature between 80° C. and 85° C. and stirred for an additional 2 hours. The reaction mixture was then adjusted to a temperature of 75° C. and purged with a stream of nitrogen for 150 minutes. The reaction mixture was then cooled to room temperature and an additional 762 g of vinyl acetate was added. The final product solution was transferred into a storage container.

The final product of Formulation 1 is a solution having the following components:

| | Concentration |
|---|---|
| Component | (% by weight) |
| 4-acetoxy-2,2,6,6-tetramethylpiperidin-1-oxyl | 19% ± 1% |
| 4-acetoxy-2,2,6,6-tetramethylpiperidin-1-ol | 1.75% ± 0.25% |
| bis((2,2,6,6-tetramethylpiperidin-1-oxyl)-4-yl) oxalate | 4% ± 1% |
| Vinyl acetate | 77.5% ± 2.5% |

### Example 2—Preparation of Formulation 2

A composition of the disclosure, hereafter referred to as Formulation 2, was prepared using the following procedure.

A reaction vessel was charged with 206 g HTEMPO, 114 g vinyl acetate, 6.18 g DMAP, and 50 g dimethyl phthalate. The resultant solution was heated to 65° C. and agitated until all of the HTEMPO was dissolved. The reaction mixture was then agitated for 30 minutes at a temperature of 50° C. The reaction mixture was then heated to 83° C. and stirred for 1 hour. Subsequently, the reaction mixture was maintained at a temperature of 75° C. and purged with a stream of nitrogen for 150 minutes. The reaction mixture was then cooled to room temperature and 635 g dimethyl phthalate were added. The final product solution was transferred into a storage container.

The final product of Formulation 2 is a solution having the following components:

| | Concentration |
|---|---|
| Component | (% by weight) |
| 4-acetoxy-2,2,6,6-tetramethylpiperidin-1-oxyl | 19% ± 1% |
| 4-acetoxy-2,2,6,6-tetramethylpiperidin-1-ol | 1.75% ± 0.25% |
| 4-hydroxy-2,2,6,6-tetramethylpiperidin-1-ol | 1.75% ± 0.25% |
| bis((2,2,6,6-tetramethylpiperidin-1-oxyl)-4-yl) oxalate | 4% ± 1% |
| Dimethyl phthalate | 74% ± 1% |

### Example 3—Preparation of Formulation 3

A composition of the disclosure, hereafter referred to as Formulation 3, was prepared using the following procedure.

A reaction vessel was charged with 206 g HTEMPO, 114 g vinyl acetate, 6.18 g DMAP, 50 g DMF. The resultant solution was heated to 65° C. to dissolve HTEMPO after which the reaction temperature was kept at 50° C. for 30 minutes. The temperature was increased to 83° C. as the reaction solution was kept stirring for 60 minutes. The resultant solution was heated to 65° C. and agitated until all of the HTEMPO was dissolved. The reaction mixture was then agitated for 30 minutes at a temperature of 50° C. The reaction mixture was then heated to 83° C. and stirred for 1 hour. Subsequently, the reaction mixture was maintained at a temperature of 75° C. and purged with a stream of nitrogen for 150 minutes to remove the acetaldehyde byproduct. The reaction mixture was then cooled to room temperature and 635 g dimethylformamide were added. The final product solution was transferred into a storage container.

The final product of Formulation 3 is a solution having the following components:

| | Concentration |
|---|---|
| Component | (% by weight) |
| 4-acetoxy-2,2,6,6-tetramethylpiperidin-1-oxyl | 19% ± 1% |
| 4-acetoxy-2,2,6,6-tetramethylpiperidin-1-ol | 1.75% ± 0.25% |
| 4-hydroxy-2,2,6,6-tetramethylpiperidin-1-ol | 1.75% ± 0.25% |
| bis((2,2,6,6-tetramethylpiperidin-1-oxyl)-4-yl) oxalate | 4% ± 1% |
| Dimethylformamide | 74% ± 1% |

### Example 4—Preparation of Formulation 4

A composition of the disclosure, hereafter referred to as Formulation 4, was prepared using the following procedure.

A reaction vessel was charged with 206 g HTEMPO, 132 g vinyl propionate, 6.18 g DMAP, and 50 g dimethyl phthalate. The resultant solution was heated to 65° C. and agitated until all of the HTEMPO was dissolved. The reaction mixture was then agitated for 30 minutes at a temperature of 50° C. The reaction mixture was then heated to 83° C. and stirred for 1 hour. Subsequently, the reaction mixture was maintained at a temperature of 75° C. and purged with a stream of nitrogen for 150 minutes to remove the reaction byproduct. The reaction mixture was then cooled to room temperature and 635 g dimethyl phthalate were added. The final product solution was transferred into a storage container.

The final product of Formulation 4 is a solution having the following components:

| | Concentration |
|---|---|
| Component | (% by weight) |
| 4-propionoxy-2,2,6,6- tetramethylpiperidin-1-oxyl | 19% ± 1% |
| 4-acetoxy-2,2,6,6-tetramethylpiperidin-1-ol | 2.5% ± 0.5% |
| 4-hydroxy-2,2,6,6-tetramethylpiperidin-1-ol | 2.5% ± 0.5% |
| bis((2,2,6,6-tetramethylpiperidin-1-oxyl)-4-yl) oxalate | 4% ± 1% |
| Dimethyl phthalate | 74% ± 1% |

### Example 5—Polymerization Inhibition of Acrylic Acid

The ability of compositions of the disclosure to inhibit the polymerization of acrylic acid was assessed via the following protocol.

250 mL round bottom flasks were charged with 30 g of freshly distilled acrylic acid and a polymerization inhibitor to be tested was individually added to each flask. Specifically, the polymerization inhibition afforded by 3.2 ppm HTEMPO, 3.2 ppm OTEMPO, 6.4 ppm Formulation 1(50% active component), and 6.4 ppm Formulation 4 (50% active component) was assessed. The samples were heated to 110° C. and agitated on a carousel. The time from the solution reaching 110° C. to when it became hazy was then recorded as the endpoint of the assay.

The samples tested and the results of the assay are summarized in Table 1, below. Results are also presented in FIG. 1.

Formulation 2 and Formulation 4 show an unexpectedly higher antipolymerant activity than 4-acetoxy-2,2,6,6-tetramethylpiperidin-1-oxyl (4-Acetoxy TEMPO), 4-acetoxy-2,2,6,6-tetramethylpiperidin-1-ol (4-acetoxy TEMPOH), 4-propionoxy-2,2,6,6-tetramethylpiperidin-1-oxyl (4-proprionate TEMPO), 4-propionoxy-2,2,6,6-tetramethylpiperidin-1-ol (4-propionate TEMPOH), 4-hydroxy-2,2,6,6-tetramethylpiperidin-1-oxyl (HTEMPO), 4-hydroxy-2,2,6,6-tetramethylpiperidin-1-ol (HTEMPOH), and 4-oxo-2,2,6,6-tetramethylpiperidin-1-oxyl (OTEMPO), alone. Furthermore, Formulations 2 and 4 demonstrate synergy, in that the combination of the polymerization inhibitor having a stable nitroxide radical with a polymerization inhibitor having a hydroxylamine produced greater polymerization inhibition than either component alone, controlling for the total dosage of the active component.

TABLE 1

| | | | | | Formulation | Formulation |
|---|---|---|---|---|---|---|
| | Formulation | | | Gelation | Performance | Performance |
| | | Weight | Conc. | Time | Over | Over |
| Inhibitor | Component | (ppm) | (mmol) | (min) | Component | HTEMPO |
| Formulation 2 | 4-acetoxy TEMPO | 2.9 | 13.6 | 272 | | 272/160 = 1.70 |
| | 4-acetoxy TEMPOH | 0.2 | 0.9 | | | |
| | HTEMPOH | 0.2 | 1.1 | | | |
| Formulation 2 w/ | 4-acetoxy TEMPO | 1.4 | 6.5 | 271 | | 271/160 = 1.69 |
| 4-acetoxy TEMPOH | 4-acetoxy TEMPOH | 1.8 | 8.4 | | | |
| | HTEMPOH | 0.2 | 1.1 | | | |
| 4-acetoxy TEMPO | | 3.2 | 15.0 | 230 | 272/230 = 1.8 | 230/160 = 1.44 |
| | | | | | (Formulation 2) | |
| 4-acetoxy TEMPOH | | 3.2 | 14.9 | 184 | 272/184 = 1.48 | 184/160 = 1.15 |
| | | | | | (Formulation 2) | |
| HTEMPOH | | 3.2 | 18.5 | 480 | 272/160 = 1.51 | 180/160 = 1.13 |
| | | | | | (Formulation 2) | |
| HTEMPO | | 3.2 | 18.6 | 460 | 272/160 = 1.70 | |
| | | | | | (Formulation 2) | |
| OTEMPO | | 3.2 | 18.8 | 94 | 272/94 = 2.89 | |
| | | | | | (Formulation 2) | |
| Formulation 4 | 4-propionate TEMPO | 2.9 | 12.7 | 356 | | 356/160 = 2.33 |
| | 4-proprionate TEMPOH | 0.2 | 0.9 | | | |
| | HTEMPOH | 0.2 | 1.1 | | | |
| 4-proprionate TEMPO | | 3.2 | 14.0 | 296 | 356/296 = 1.20 | 296/160 = 1.85 |
| | | | | | (Formulation 4) | |
| 4-proprionate TEMPOH | | 3.2 | 14.0 | 310 | 356/310 = 1.15 | 310/160 = 1.94 |
| | | | | | (Formulation 4) | |

### Example 6—Polymerization Inhibition of Vinyl Acetate in Acetic Acid

The ability of compositions of the disclosure to inhibit the polymerization of vinyl acetate in the presence of acetic acid was assessed via the following protocol.

An 8 ounce jar was charged with 0.0125 g HTEMPO, OTEMPO, 0.0250 g Formulation 2 (50% active component), or 0.0250 g Formulation 4 (50% active component). 0.125 g benzoyl peroxide was added to each jar, followed by 40 wt % acetic acid in free vinyl acetate solution to a final mass of 250 g. A batch of twelve pressure tubes were charged with 10 g of the above solution with a stir bar. The pressure tubes were purged with nitrogen for 2 minutes, and each tube was then sealed immediately to maintain a nitrogen headspace. The tubes were loaded into a heating block that had been preheated to 75° C. After 20 minutes, and every 30 minutes thereafter, two tubes were retrieved from the block and the polymerization reaction quenched by cooling in an ice bath. The cooled polymer solutions were immediately diluted with tetrahydrofuran if necessary. The polymer content was then assessed for each dilution. The time taken to reach a soluble polymer content greater than 2% was used as the endpoint for determining gelation of the sample.

The samples tested and the results of the assay are summarized in Table 2, below. Results are also presented in FIG. 2.

Formulation 2 and Formulation 4 show an unexpectedly higher antipolymerant activity than 4-acetoxy-2,2,6,6-tetramethylpiperidin-1-oxyl (4-Acetoxy TEMPO), 4-acetoxy-2,2,6,6-tetramethylpiperidin-1-ol (4-acetoxy TEMPOH), 4-propionoxy-2,2,6,6-tetramethylpiperidin-1-oxyl (4-proprionate TEMPO), 4-propionoxy-2,2,6,6-tetramethylpiperidin-1-ol (4-propionate TEMPOH), 4-hydroxy-2,2,6,6-tetramethylpiperidin-1-oxyl (HTEMPO), and 4-oxo-2,2,6,6-tetramethylpiperidin-1-oxyl (OTEMPO), alone. Furthermore, Formulations 1 and 4 demonstrate synergy, in that the combination of the polymerization inhibitor having a stable nitroxide radical with a polymerization inhibitor having a hydroxylamine produced greater polymerization inhibition than either component alone, controlling for the total dosage of the active component.

TABLE 2

| | | | | | Formulation | Formulation |
|---|---|---|---|---|---|---|
| | Formulation | | | Gelation | Performance | Performance |
| | | Weight | Conc. | Time | Over | Over |
| Inhibitor | Component | (ppm) | (mmol) | (min) | Component | HTEMPO |
| Formulation 2 | 4-acetoxy TEMPO | 45 | 210 | 100 | | 100/80 = 1.25 |
| | 4-acetoxy TEMPOH | 3 | 14 | | | |
| | HTEMPOH | 3 | 17 | | | |
| Formulation 2 w/ | 4-acetoxy TEMPO | 23 | 107 | 100 | | 100/80 = 1.25 |
| 4-acetoxy TEMPOH | 4-acetoxy TEMPOH | 27 | 126 | | | |
| | HTEMPOH | 2 | 12 | | | |
| 4-acetoxy TEMPO | | 50 | 232 | 90 | 100/90 = 1.11 | 90/80 = 1.13 |
| | | | | | (Formulation 2) | |
| 4-acetoxy TEMPOH | | 50 | 289 | 90 | 100/90 = 1.11 | 90/80 = 1.13 |
| | | | | | (Formulation 2) | |
| HTEMPO | | 50 | 291 | 80 | 100/80 = 1.25 | |
| | | | | | (Formulation 2) | |
| OTEMPO | | 50 | 294 | 80 | 100/80 = 1.25 | |
| | | | | | (Formulation 2) | |
| Formulation 4 | 4-propionate TEMPO | 56 | 197 | 100 | | 100/80 = 1.25 |
| | 4-proprionate TEMPOH | 3 | 14 | | | |
| | HTEMPOH | 3 | 17 | | | |
| 4-proprionate TEMPO | | 50 | 219 | 90 | 100/90 = 1.11 | 90/80 = 1.13 |
| | | | | | (Formulation 4) | |
| 4-proprionate TEMPOH | | 50 | 218 | 90 | 100/90 = 1.11 | 90/80 = 1.13 |
| | | | | | (Formulation 4) | |

All of the compositions and methods disclosed and claimed herein can be made and executed without undue experimentation in light of the present disclosure. While this invention may be embodied in many different forms, there are described in detail herein specific preferred embodiments of the invention. The present disclosure is an exemplification of the principles of the invention and is not intended to limit the invention to the particular embodiments illustrated. In addition, unless expressly stated to the contrary, use of the term “a” is intended to include “at least one” or “one or more.” For example, “a compound” is intended to include “at least one compound” or “one or more compounds.”

Any ranges given either in absolute terms or in approximate terms are intended to encompass both, and any definitions used herein are intended to be clarifying and not limiting. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges (including all fractional and whole values) subsumed therein.

Any composition disclosed herein may comprise, consist of, or consist essentially of any element, component and/or ingredient disclosed herein or any combination of two or more of the elements, components or ingredients disclosed herein.

Any method disclosed herein may comprise, consist of, or consist essentially of any method step disclosed herein or any combination of two or more of the method steps disclosed herein.

The transitional phrase “comprising,” which is synonymous with “including,” “containing,” or “characterized by,” is inclusive or open-ended and does not exclude additional, un-recited elements, components, ingredients and/or method steps.

The transitional phrase “consisting of” excludes any element, component, ingredient, and/or method step not specified in the claim.

The transitional phrase “consisting essentially of” limits the scope of a claim to the specified elements, components, ingredients and/or steps, as well as those that do not materially affect the basic and novel characteristic(s) of the claimed invention.

Unless specified otherwise, all molecular weights referred to herein are weight average molecular weights and all viscosities were measured at 25° C. with neat (not diluted) polymers.

As used herein, the term “about” refers to the cited value being within the errors arising from the standard deviation found in their respective testing measurements, and if those errors cannot be determined, then “about” may refer to, for example, within 5% of the cited value.

Furthermore, the invention encompasses any and all possible combinations of some or all of the various embodiments described herein. It should also be understood that various changes and modifications to the presently preferred embodiments described herein will be apparent to those skilled in the art. Such changes and modifications can be made without departing from the spirit and scope of the invention and without diminishing its intended advantages. It is therefore intended that such changes and modifications be covered by the appended claims.

What is claimed is:

1. A composition for inhibiting monomer polymerization comprising:
a first inhibitor compound comprising a stable nitroxide radical; and
a second inhibitor compound comprising a hydroxylamine.

2. The composition of claim 1, wherein the first inhibitor compound is of formula (I): 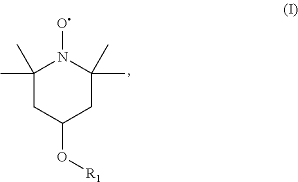
wherein R₁ is selected from H, C₁-C₂₂ alkyl, C₁-C₂₂ alkenyl, C₁-C₂₂ alkynyl, C₁-C₂₂ cycloalkyl, aryl, —C₁-C₂₂ alkylene aryl, —C(O)(C₁-C₂₂ alkyl), —C(O)(C₁-C₂₂ alkenyl), —C(O)(C₁-C₂₂ alkynyl), —C(O)(C₁-C₂₂ cycloalkyl), —C(O)(aryl), and —C(O)(C₁-C₂₂ alkylene aryl), wherein the cycloalkyl and aryl are optionally substituted with one or more C₁-C₂₂ alkyl, C₁-C₂₂ alkenyl, C₁-C₂₂ alkynyl, or aryl.

3. The composition of claim 1, wherein R₁ is selected from —C(O)(C₁-C₂₂ alkyl), —C(O)(C₁-C₂₂ alkenyl), —C(O)(C₁-C₂₂ alkynyl), —C(O)(C₁-C₂₂ cycloalkyl), —C(O)(aryl), and —C(O)(C₁-C₂₂ alkylene aryl), wherein the cycloalkyl and aryl are optionally substituted with one or more C₁-C₂₂ alkyl, C₁-C₂₂ alkenyl, C₁-C₂₂ alkynyl, or aryl.

4. The composition of claim 1, wherein the second inhibitor compound is of formula (II): 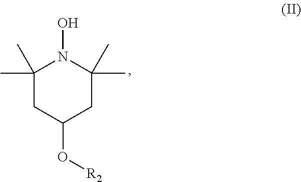
wherein R₂ is selected from H, C₁-C₂₂ alkyl, C₁-C₂₂ alkenyl, C₁-C₂₂ alkynyl, C₁-C₂₂ cycloalkyl, aryl, —C₁-C₂₂ alkylene aryl, —C(O)(C₁-C₂₂ alkyl), —C(O)(C₁-C₂₂ alkenyl), —C(O)(C₁-C₂₂ alkynyl), —C(O)(C₁-C₂₂ cycloalkyl), —C(O)(aryl), and —C(O)(C₁-C₂₂ alkylene aryl), wherein the cycloalkyl and aryl are optionally substituted with one or more C₁-C₂₂ alkyl, C₁-C₂₂ alkenyl, C₁-C₂₂ alkynyl, or aryl.

5. The composition of claim 4, wherein R₂ is selected from —C(O)(C₁-C₂₂ alkyl), —C(O)(C₁-C₂₂ alkenyl), —C(O)(C₁-C₂₂ alkynyl), —C(O)(C₁-C₂₂ cycloalkyl), —C(O)(aryl), and —C(O)(C₁-C₂₂ alkylene aryl), wherein the cycloalkyl and aryl are optionally substituted with one or more C₁-C₂₂ alkyl, C₁-C₂₂ alkenyl, C₁-C₂₂ alkynyl, or aryl.

6. The composition of claim 1, wherein the first inhibitor compound is selected from the group consisting of:

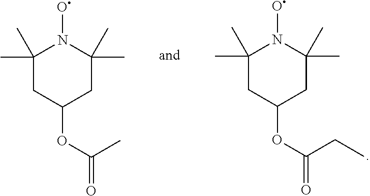

7. The composition of claim 1, wherein the second inhibitor compound is selected from the group consisting of:

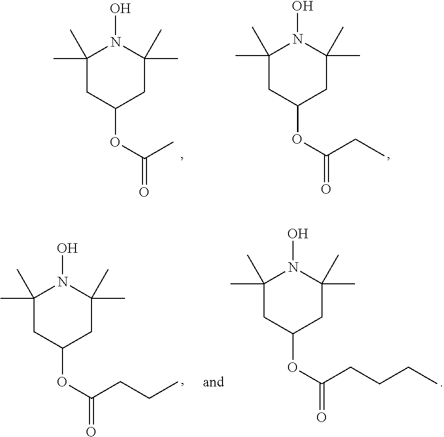

8. The composition of claim 1, wherein the first inhibitor compound is present in the composition at a concentration of about 0.01% by weight to about 80% by weight.

9. The composition of claim 1, wherein the second inhibitor compound is present in the composition at a concentration of about 0.01% by weight to about 50% by weight.

10. The composition of claim 1, wherein a mole ratio of the first inhibitor compound to the second inhibitor compound is about 100:1 to about 1:100.

11. The composition of claim 1, wherein the composition further comprises one or more additional compounds selected from the group consisting of: 2,2,6,6-tetramethylpiperidin-1-oxyl; 2,2,6,6-tetramethylpiperidin-1-ol; 4-hydroxyl-2,2,6,6-tetramethylpiperidin-1-oxyl; 4-hydroxy-2,2,6,6-tetramethylpiperidin-1-ol; 4-oxo-2,2,6,6-tetramethylpiperidin-1-oxyl; 4-oxo-2,2,6,6-tetramethylpiperidin-1-ol; 4-acetoxy-2,2,6,6-tetramethylpiperidin-1-oxyl; 4-acetoxy-2,2,6,6-tetramethylpiperidin-1-ol; 4-propionoxy-2,2,6,6-tetramethylpiperidin-1-oxyl; 4-propionoxy-2,2,6,6-tetramethylpiperidin-1-ol; and bis((2,2,6,6-tetramethylpiperidin-1-oxyl)-4-yl) oxalate.

12. The composition of claim 1, further comprising an ethylenic unsaturated monomer.

13. The composition of claim 1, wherein the composition further comprises an acid or an acetaldehyde.

14. A method of inhibiting polymerization of a monomer, the method comprising:
adding the composition of claim 1 to the monomer.

15. The method of claim 14, wherein the monomer is provided within a solution, optionally wherein the solution further comprises one or more additional components selected from the group consisting of an acid, an organic solvent, water, and any combination thereof.

16. The method of claim 14, wherein the composition is added to the monomer such that a concentration of the first inhibitor compound is about 0.1 ppm to about 10,000 ppm and/or a concentration of the second inhibitor compound is about 0.1 ppm to about 10,000 ppm.

17. A process for preparing a compound of formula (III): 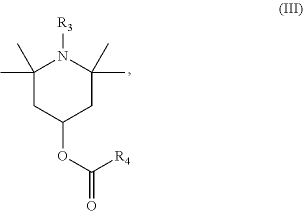
wherein:
R₃ is —O or —OH; and
R₄ is C₁-C₂₂ alkyl, C₁-C₂₂ alkenyl, C₁-C₂₂ alkynyl, C₁-C₂₂ cycloalkyl, aryl, and C₁-C₂₂ alkylene aryl, wherein the cycloalkyl and aryl are optionally substituted with one or more C₁-C₂₂ alkyl, C₁-C₂₂ alkenyl, C₁-C₂₂ alkynyl, or aryl, comprising:
treating a compound of formula (IIIa):
with a compound of formula (IIIb): 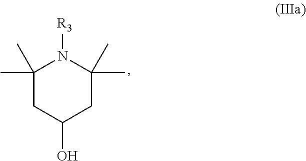 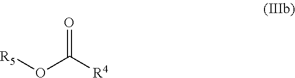
wherein R₅ is C₁-C₂₂ alkyl or C₁-C₂₂ alkenyl,
within a solution, to afford the compound of formula (III).

18. The process of claim 17, wherein the compound of formula (IIIa) is treated with the compound of formula (IIIb) in the presence of a catalyst and heat.

19. The process of claim 17, further comprising purging the solution with a stream of nitrogen.

20. The process of claim 17, wherein the step of purging the solution with a stream of nitrogen is performed concurrently with the step of treating a compound of formula (IIIa) with a compound of formula (IIIb) to afford the compound of formula (III), or wherein the step of purging the solution with a stream of nitrogen is performed after the step of treating a compound of formula (IIIa) with a compound of formula (IIIb) to afford the compound of formula (III).
